(19) United States
(12) Patent Application Publication
(10) Pub. No.: US 2024/0102053 A1
(43) Pub. Date: Mar. 28, 2024

(54) Method for Producing Genetically Engineered Cells

(71) Applicant: Cytosurge AG, Glattbrugg (CH)

(72) Inventors: Tobias A. Beyer, Zollikerberg (CH); Michael Gabi, Glattbrugg (CH); Stephan Jud, Meilen (CH); Matyas Vegh, Zürich (CH); Maria Mila, Zürich (CH); Samuel Riedmann, Bonstetten (CH); Paul Monnier, Zürich (CH); Pascal Behr, Eglisau (CH)

(21) Appl. No.: 18/276,061

(22) PCT Filed: Feb. 4, 2022
(86) PCT No.: PCT/EP2022/052779

§ 371 Date: Aug. 7, 2023

(30) Foreign Application Priority Data
Feb. 9, 2021 (CH) 00119/21

(51) Int. Cl. C12N 15/90; C12N 9/22; C12N 15/11; C12N 15/89

(52) U.S. Cl. CPC C12N 15/907; C12N 9/22; C12N 15/111; C12N 15/89; C12N 2310/20

(57) ABSTRACT

Disclosed is a method for producing genetically engineered cells in vitro. The method includes direct injection of a genome editing composition into a cell nucleus of a cell. The genome editing composition includes at least one Cas protein and at least one gRNA molecule to target a distinct genomic location. The injection is performed with a microelectromechanical systems injection chip including a cantilever. The cantilever includes a microchannel being in fluid communication with a nanosyringe, and wherein direct injection includes providing a fluid communication between the microchannel and the nucleus of the cell by insertion of the nanosyringe into the nucleus of the cell and injecting the genome editing composition via the microchannel through the nanosyringe into the nucleus of the cell.

### CROSS-REFERENCE TO RELATED APPLICATIONS

This application is the United States national phase of International Application No. PCT/EP2022/052779 filed Feb. 4, 2022, and claims priority to Swiss Patent Application No. 00119/21 filed Feb. 9, 2021, the disclosures of each of which are hereby incorporated by reference in their entireties.

### BACKGROUND OF THE INVENTION

### Field of the Invention

The present invention relates to genetic engineering of cells, in particular to a method for producing genetically engineered cells.

### Description of Related Art

Genome engineering tools are particularly based on programmable nucleases (PN) that include meganuclease 3, zinc-finger nucleases (ZNFs) transcription activator-like effector nucleases (TALENs) and most prominent the class of Clustered Regularly Interspaced Short Palindromic Repeats (CRISPR)/CRISPR-associated (Cas) nucleases.

Inducing DNA strand breaks (single-strand breaks [SSB] and double-strand breaks [DSB]) with PN elicits a cellular DNA repair response to reverse the damage. In most cases, the DSB is repaired by re-ligation of the DNA. This often leads to alteration of the sequence and subsequently a non-functional gene product. Alternatively, templated repair from a separate DNA donor molecule can occur through homology-directed repair (HDR). These DNA repair processes are harnessed by genome editing to introduce desired genomic alteration.

The advancement of genome engineering, personalized medicine and synthetic biology requires numerous and complex, genetically engineered cell lines. Over the past decades these cells lines have become crucial for biopharmaceutical research for enabling a better understanding of basic biology, model diseases and develop novel, personalized therapeutics. Therefore, they are in need for numerous engineered cell lines to enable their research and achieve these goals.

Existing manufacturing methods to generate engineered cell lines are based on bulk methods to deliver DNA, RNA, proteins, or combinations of these macromolecules to millions of cells. Depending on the nature of the cargo, delivery methods include, but are not limited to viral vectors, lipid-based vesicles, or physical methods such as electroporation. Transfection rates vary from 0-99% depending on cell type, transfection method and/or the cargo delivered. In addition, all these approaches can have toxic effects on cells leading to necrosis or programmed cell death. If the transfection rate is sufficiently high (>90%), polyclonal bulk cultures can be used for specific applications.

However, in fundamental biology and, for translational applications, monoclonal cell lines are required. After the successful delivery of these macromolecules a tedious selection process is necessary to identify and isolate the altered cells to generate monoclonal cell lines. This can be achieved by adding selectable markers, such as a plasmid coding for a fluorescent protein or a drug resistance to purify cells by FACS or drug selection. The selected cells form clonal colonies and subsamples of individual colonies are collected to test whether the desired modification is present. Correctly modified cell clones are subjected to several rounds of serial dilutions to insure true monoclonality. Overall, this process is tedious, time consuming and does not scale well. The scaling issue is even further aggravated, when performing multiple modification in the same cells, as the generation time roughly scales linearly with every additional modification/edit. The generation time for a cell line with n modifications is n times 10 weeks. Furthermore, due to low transfection efficiencies, many known processes do not provide sufficient control of the actual concentrations of the required engineering components, such as CRISPR components in the target cells.

### SUMMARY OF THE INVENTION

It is the general object of the present invention to advance the state of the art of methods for producing genetically engineered cells and preferably to overcome the disadvantages of the prior art fully or partly. In favorable embodiments, a method for producing genetically engineered cells is provided, which can be completed in significantly less time than methods known in the prior art. In further favorable embodiments method for producing genetically engineered cells is provided, which enables a more convenient and/or faster process to generate monoclonal cell assemblies.

### BRIEF DESCRIPTION OF THE DRAWINGS

FIG. 1 shows a schematic illustration of a classical genetic engineering approach. Cells are transfected with classical transfection methods, expanded for 0-72 h (1) and positive cells (*) are selected by either FACS or drug resistance screening (2), while dead cells are discarded (3). The selected cells are then expanded (4) and analyzed. Positive cell clones are further cultivated (5) and subjected to several rounds of serial dilutions, which are necessary to ensure monoclonality (6) and (7). The overall process to produce a monoclonal cell lines takes usually around 10 weeks.

FIG. 2 shows the direct injection of genome editing composition 3 into cell nucleus 2 of single isolated cell 1 according to an embodiment of the invention via the shown cantilevered nanosyringe.

FIG. 3 shows another embodiment of the method according to the invention. A cell preparation comprising a plurality of cells is provided and in step (1) direct injection of the gene composition is performed into one or more cells of the cell preparation. After a certain time, one of the cells having been injected with the genome editing composition (indicated by the black dot) is selected and separated from the cell preparation by means of microelectromechanical systems transport chip comprising a cantilever with an opening and a microchannel extending through the cantilever to the opening. By application of a negative pressure to the microchannel, the cell is partially sucked into the opening and adheres to the cantilever.

FIG. 4a) and b) show different embodiments of a microelectromechanical systems injection chip comprising a cantilever 4 with microchannel 5, which is in fluidic communication with nanosyringe 6. Nanosyringe 6 has in the embodiment of FIG. 4a) a pyramidal structure as described herein. Opening 7, which is in fluidic communication with microchannel 5 is not arranged on apex 8, but on the shell surface of the pyramidal structure. In FIG. 4b, nanosyringe 6 comprises hollow tubular body 9, which is a sharp tip and whose opening is in fluidic communication with microchannel 5. FIG. 4c) shows a microelectromechanical systems transport chip with cantilever 4′ and opening 7′ which is in fluidic communication with microchannel 5′. As can be seen, opening 7′ is arranged at a flat surface, thereby avoiding sharp edges which may damage the cell upon application of a negative pressure to microchannel 5′.

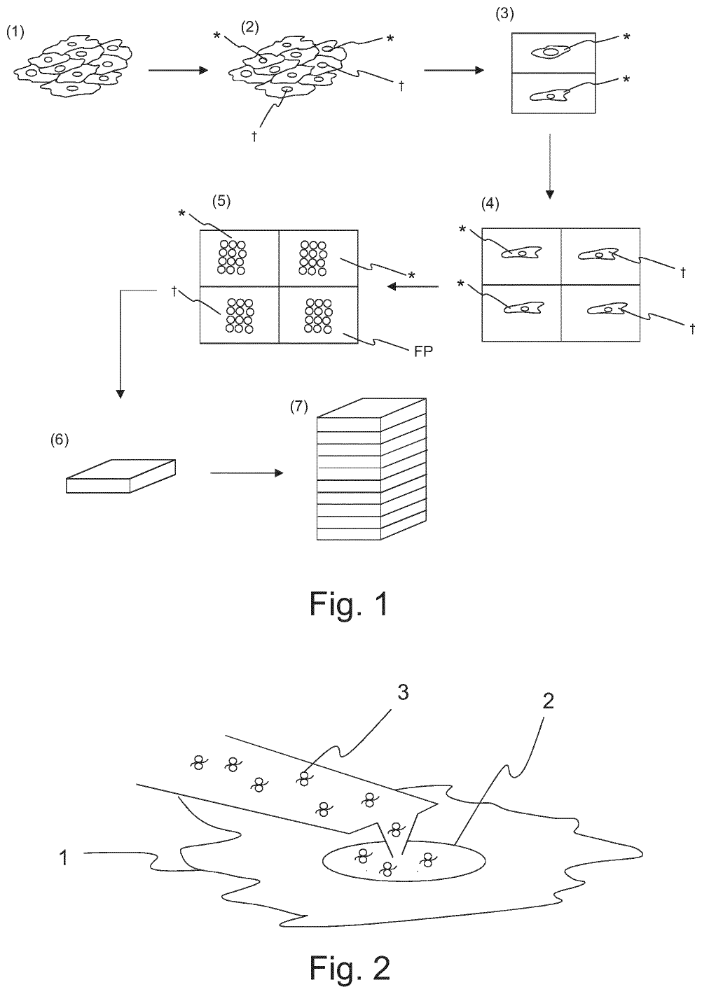

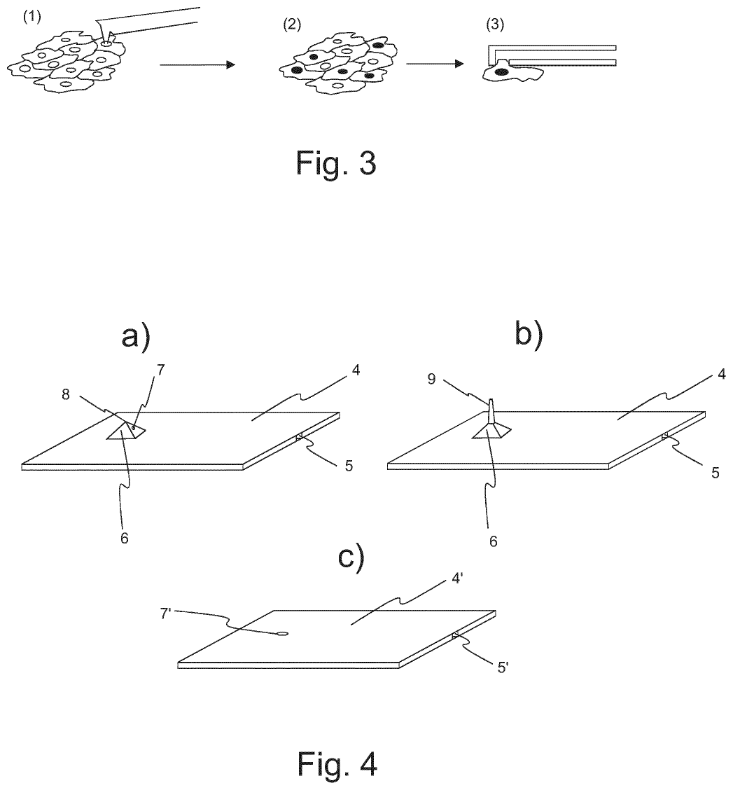

### DETAILED DESCRIPTION

In a first aspect, the general objective is achieved by a method for producing genetically engineered cells in vitro as described herein. The method comprises the direct injection of a genome editing composition into the nucleus of a cell, preferably an adherent cell, i.e. a cell adhering to a surface. The genome editing composition comprises at least one Cas protein and at least one gRNA molecule directed to a genomic location to be edited. The gRNA typically consists of two functional parts: a hairpin structure responsible to bind the Cas protein and a sequence complementary to the DNA to be targeted. The injection is performed with a microelectromechanical systems injection chip comprising a cantilever, which may in particular be an AFM cantilever, wherein the cantilever comprises a microchannel being in fluid communication with a nanosyringe of the cantilever. Direct injection comprises providing a fluid communication between the microchannel and the nucleus of the cell by inserting the nanosyringe into the nucleus of the cell and injecting the genome editing composition via the microchannel through the nanosyringe into the nucleus of the cell. The method according to the invention enables to directly introduce the genome editing composition into the nucleus of a single cell, which amongst others allows to dispense with engineered Cas proteins, such as for example Cas proteins comprising NLS sequences (nuclear localization sequence). In contrast to bulk transfection to thousands of cells, a much more reliable and controllable process is provided, as the stoichiometry can be accurately controlled. In addition, the volume of the genome editing composition being injected into each nucleus can be precisely determined. The single cell itself can then be used as an origin source for expansion in order to provide a monoclonal cell line, which is then intrinsically monoclonal. Therefore, tedious selection and analysis processes of cells are avoided. The injection as such is accurately controllable due to the force-feedback penetration of the cell by the cantilevered nanosyringe, thereby avoiding cell damage and thus cell death, which ultimately increases the amount of successfully engineered cells. In general, the cantilever bending can be controlled by a laser beam as it is for example common in AFM in combination with a photodiode configured for receiving the laser beam being reflected from the cantilever.

Furthermore, the use of microelectromechanical systems injection chip according to the invention allows to perform the injection step itself in short time without loss of control, i.e. within 1 s to 60 s.

It is understood that a gRNA can comprise a single RNA with a scaffold to bind the Cas protein and a spacer complementary to the targeted DNA region. Alternatively, the gRNA can comprise a crRNA, complementary to the target DNA and a tracrRNA responsible for Cas binding. It is understood that the crRNA and tracrRNA must be annealed prior binding to the Cas protein.

A Cas protein is understood as a CRISPR associated protein. Upon binding to a gRNA, the Cas protein can be directed to a defined genomic location. Cas proteins can include nucleases inducing SS (Nickase) and DS breaks and catalytic dead nucleases. Cas proteins can be engineered to contain base editor activities, transcriptional activation or repressor domains and other activities of interest. It is understood that the claim is not limited to a specific Cas protein from a specific species (e. g. Cas9) but extends to other types of Cas proteins (Class 1, Type I, III, IV, Class 2, Type II, V, VI)

It is further understood that for each DNA sequence to be targeted, there is an individual pair of a gRNA complementary to the DNA bound to a Cas protein. Therefore, to target several genomic loci at once, there must be at least one gRNA/Cas complex to be present that includes a gRNA complementary to the target site.

A nanosyringe is understood as a structural feature comprising tip with an opening allowing for injecting volumes in the range of less than 1 nL, preferably less than 1 pL within 1-60 s and an applied pressure of 1-5000 mbar to an aqueous solution e.g. genome editing composition. An opening in the sub-micrometer range (nanometer range) is suitable for fulfilling these conditions, e.g. an opening of a diameter of 10-1500 nm, preferably between 10 to 999 nm.

In some embodiments, the opening of the nanosyringe has a size, particularly a diameter, of less than 10 μm, preferably less than 5 μm. In particular, the opening may have a size, particularly a diameter, of 10 nm to 5 μm, e.g. 0.1 μm to 1.5 μm.

In some embodiments, the nanosyringe may have a pyramidal structure, wherein the opening is arranged on the shell surface of the pyramidal structure. Such nanosyringes are advantageous as the apex of the pyramidal structure serves as a sharp puncturing tip thereby providing for a well-defined puncturing of the cell.

In some embodiments, the nanosyringe may comprise a hollow tubular body, preferably at least partially having a cylindrical shape, configured as nanosyringe tip.

In some embodiments, the cantilever and/or the microchannel, in particular the walls of the microchannel are coated with an antifouling agent, such as an organopolysiloxane, in particular a halogenated organopolysiloxane. Suitable antifouling agent may be Pacram® or Sigmacote®.

Typically, the genome editing composition may be an aqueous solution.

In some embodiments, the method further comprises the step of expanding the cell produced to generate a first monoclonal cell culture. Expanding can for example be performed in a single well. It is understood that a cell culture comprises a plurality of cells, i.e. it may be considered as a cell line. The method according to the invention allows for accurately selection and injection of the corresponding cell. If such as cell after injection is expanded, for example by cell cultivation under suitable conditions, the resulting cell culture is as such monoclonal. The method allows therefore for a streamlined production of genetically modified cells within only 2 to 4 weeks.

In some embodiments, the cells of the first monoclonal cell culture, i.e. the cell culture obtained by expanding the cell produced, are divided into at least two, in particular two or three sub-groups, such as a first sub-group, a second-subgroup and optionally a third sub-group. In particular, dividing the cell culture may be performed after 10-20 population doublings of the cell produced. In certain embodiments, the cells of the first monoclonal cell culture are divided into only two, or only three sub-groups.

In some embodiments, the first sub-group may comprise 5% to 20% of the cells of the first monoclonal cell culture. In some embodiments, the second sub-group may comprise 60% to 90% of the cells of the first monoclonal cell culture. In some embodiments, the third sub-group may comprise 5% to 20% of the cells of the first monoclonal cell culture.

In some embodiments, the cells of the first sub-group, and in particular their DNA sequence, are analyzed by sequencing. In particular, the cell DNA may first be liberated, for example by chemical and/or enzymatic treatment of the cells. In certain embodiments, the liberated DNA is amplified by PCR. In particular, ssDNA oligonucleotides may be used for PCR which are located upstream and downstream of genomic location to be edited. The liberated and amplified DNA is then analyzed by sequencing. In some embodiments, the liberated DNA, respectively the amplified DNA is sequenced by Sanger sequencing.

In some embodiments, the obtained sequences are compared to an expected unmodified sequence to identify edits. Edits may further be characterized by a suitable computer implemented method.

In some embodiments, the cells of the second sub-group are expanded further to generate a second monoclonal cell culture, in particular in wells of a well plate. This step is preferably performed while the cells of the first sub-group are analyzed. As the skilled person understands, expansion is performed in a suitable cell culture medium. The expansion of the cells of the second sub-group may be performed for 2 to 12 population doublings, respectively for 2 to 4 days. The sequencing results obtained from analyzing the cells of the first sub-group may be used to identify the genotype of the cells of the second sub-group. In certain embodiments, cells containing the desired mutations are preserved, for example by cryopreservation. The cryopreservation medium may for example consist of either the regular growth medium containing 1-30% DMSO or a specialized commercial cryopreservation medium (e. g. Cryostore, Sigma). In some embodiments, a certain amount of cells which are unmodified, i.e. which do not contain the desired mutation, are also preserved, in particular cryopreserved. This has the advantage that a set of control cells which have undergone the same treatment as the modified cells is obtained, which is beneficial for control experiments and thus reduces errors in data interpretation in experiments on the obtained second monoclonal cell culture.

Embodiments in which the cells of the first monoclonal cell culture, i.e. the cell culture obtained by expanding the cell produced, are divided into two sub-groups, i.e. in the first and second sub-group as described above significantly increase the efficiency of cell line generation, because the first sub-group is analyzed while the cells are further expanded and preserved. Thereby it is ensured that only cells which have undergone the desired genetic modification are preserved.

In some embodiments, the cells of the third sub-group are expanded further to produce a third monoclonal cell culture, in particular in wells of a well plate. It is understood that the cells of the third sub-group are expanded independently and separately from the cells of the second sub-group. As the skilled person understands, expansion is performed in a suitable cell culture medium. The expansion of the cells of the second sub-group may be performed for 2 to 12 population doublings, respectively for 2 to 4 days. The cryopreservation medium may for example consist of either the regular growth medium containing 1-30% DMSO or a specialized commercial cryopreservation medium (e. g. Cryostore, Sigma). The expanded cells of the third sub-group may be preserved, in particular cryopreserved. The expanded cells of the third sub-group serve as backup cells for the expanded cells of the second sub-group. Thus, accidental loss of the expanded cells of the second sub-group is avoided and backup cells can readily be provided.

Dividing the expanded cells of the first monoclonal cell culture has the advantage that the time required for producing monoclonal genetically modified cell lines is reduced to around only 2 to 6 weeks as compared to prior art methods, which require between 8 to 16 weeks and are additionally much more cumbersome. Furthermore, the number of population doublings, e.g. the number of doublings without control analysis, is reduced, which results in a more reliable method, because each additional doubling increases the risk of genetic drift.

In some embodiments, prior to the step of direct injection, a cell preparation comprising a plurality of cells, in particular, 1 to 1000 cells, is provided and direct injection of the gene composition is performed into one or more cells, for example direct injection of the gene composition is performed into up to 10%, 20%, 30%, 40% 50%, e.g. 60%, e.g. 70%, e.g. 80%, e.g. 90%, e.g. 95%, e.g. 100%, of the cells of the cell preparation. After direct injection of the genome editing composition, a single cell having been injected with the genome editing composition is selected and separated from the cell preparation and optionally subsequently individually expanded to generate the first monoclonal cell culture. Such embodiments have the advantage that due to the relatively fast injection step, multiple different monoclonal cell lines can be produced. While the injection is usually performed in series, after separation of the cells, specific single cells can be separated and optionally provided into single wells, and then serve as the origin for a monoclonal cell culture by expansion. Thus, within a significantly decreased process time, different monoclonal cell lines can be generated. It is noted that the time limiting step in such a process is not the injection, but the expansion step, which can be performed in parallel for multiple different monoclonal cell lines.

In certain embodiments, selection and separation of the single cell includes moving the single cell by means of microelectromechanical systems transport chip comprising a cantilever with an opening and a microchannel extending through the cantilever to the opening, wherein moving is performed by applying a negative pressure with respect to the environment to the microchannel such that the single cell adheres to the cantilever and moving the cantilever as a whole together with the adhered cell. Moving may be performed by contacting the single cell with the opening and applying a negative pressure. Typically, the cantilever of the microelectromechanical systems transport chip does not comprise sharp edges or tips, particularly at the opening, as compared to the cantilever of the microelectromechanical systems injection chip. Thus, the cantilever of the microelectromechanical systems transport chip may comprise a flat, elongated element or surface containing the opening. Upon application of a negative pressure, the cell to be separated can partially be sucked into the opening upon which it adheres to the cantilever. The cantilever can then be moved, particularly with respect to the remaining cells of the cell culture, in order to separate the selected cell. The cell can for example be transported, preferably automatically transported, into a separate vessel, such as a single well. In a subsequent step, the selected and separated cell can be expanded to produce a monoclonal cell culture. Such embodiments have the advantage that the microelectromechanical systems transport chip is readily combinable with the microelectromechanical systems injection chip, i.e. the same automatic elements and features can be used and that cell selection can be accurately performed in a time saving and gentle manner enabling to avoid cell damage.

In some embodiments, the adherent cell may first be detached before moving from the neighboring cells and the culture vessel by localized chemical and or enzymatic treatment (e. g. by trypsin, EDTA). These solutions can be delivered locally by the microelectromechanical systems transport chip.

In some embodiments, prior to direct injection of the genome editing composition into the nucleus of the cell, a single cell is isolated, particularly in a separate well. Thus, in contrast to the embodiment in which one or more of a plurality of cells is provided and direct injection is performed on a given cell of the corresponding cell preparation, a single cell is first isolated and separated prior to direct injection.

This is possible, because due to direct injection according to the invention, the success rate of genetic engineering is much higher than with bulk transfection methods, as the genome editing composition is directly introduced into the nucleus of the cell under highly controllable conditions. Thus, single cells can be used as a starting point, which can be engineered and then expanded to generate monoclonal cell lines. The isolation can for example be performed using a microelectromechanical systems transport chip as disclosed above. In some embodiments, isolating a single cell can be conducted with a cell printing device which deposits a single cell in a separate well, in particular in the center of a separate well. As it is readily understood, isolating a single cell can in general be repeated multiple times, such that multiple cells are isolated, each in a separate well. Thus, each well of well plate with 12, 24, 48 or 96 wells may for example contain a single isolated cell.

In some embodiments, the isolated single cell(s) are maintained in a growth medium for a certain recovery time interval, for example for 2 to 24 h. In particular, the isolated single cell(s) may be maintained at a temperature of between 4° C. and 37° C. and/or at 0-95% humidity and/or at a CO₂ concentration of 0.1 vol % to 10 vol %. In this period, the isolated single cells may recover before direct injection of the genome editing composition into their nucleus. Preferably, monoclonality may be verified before the injection.

The microelectromechanical systems injection chip and/or the microelectromechanical systems transport chip may for example be made from Si, SiO₂ or Si₃N₄.

In some embodiments, the genome editing composition further comprises transfection markers and/or a plasmid or mRNA encoding for a transfection marker. A suitable transfection marker enables to determine if transfection has been successful and provides therefore for a direct control mechanism over the method according to the invention. Furthermore, it may also simplify the selection of a cell to be separated. In certain embodiments, the transfection marker may be a fluorescent transfection marker or luciferase enzymes. Suitable fluorescent transfection markers are GFP, RFP, YFP, BFP, Rhodamine and the like. The transfection marker may in some embodiments be present in a concentration of 0.5 ng/μL to 250 ng/μL, particularly of 0.5 ng/μL to 100 ng/μL.

In some embodiments, transfection is monitored by scanning the cell into whose nucleus the genome editing composition has been injected, respectively the cells originating from this cell by expansion. In certain embodiments, monitoring is conducted with a microscope, in particular an imaging system, such as the EVOS M7000. Monitoring may be conducted at predetermined points in time or within regular time intervals. For example, monitoring may be conducted on day 1, day 2, day 4 and day 10.

In some embodiments, the genome editing composition comprises a plurality of Cas proteins and a plurality of gRNA molecules. An individual gRNA is targeted to a genomic location by sequence complementarity. The gRNA is complexed with one Cas protein and can thereby guide the Cas protein to the said genomic location. The nuclease activity of the Cas protein is subsequently cleaving the targeted genomic location. Depending on the nature of the Cas protein the nuclease can induce SS (single strand) or DS (double strand) DNA breaks.

In some embodiments, the Cas protein can carry a mutation to render its nuclease domain inactive. In this case, the Cas protein can be fused with other proteins or protein domains to add a specific activity. This can be, but not limited to a transcriptional activation or repression domains, base editing functions, a fluorescent domain, or other enzymatic activities.

For optimal binding of the gRNA to the Cas protein a stoichiometry of 10:1 to 1:10, in particular 1:1 to 1:10, is preferred.

Typically, the genome editing composition contains in general at least one individual gRNA molecule complexed to one Cas protein thereby targeting one distinct genomic entity. In some embodiments, the genome editing composition may comprise 1-500 individual gRNA/Cas complexes. These can be injected simultaneously to target 1-500 different genomic loci. Such embodiments allow for multiple gene editing events in a single cell upon a single injection step. Thus, multiple genetic editing events are performed in a time saving manner because in contrast to serial bulk transfection, the required agents can be selectively injected in a single step.

In some embodiments, the genome editing composition further comprises a buffer composition with one or more buffers with a pH range of 6 to 9, and/or inorganic salt mixtures, particularly aqueous solutions, such as NaCl, KCl, MgCl₂, CaCl₂ and mixtures thereof. The salt mixtures are configured to match the cell specific internal osmotic pressure. Suitable buffers may be selected from one or more of PBS, HEPES, Tris/HCl, Tris/HCl/EDTA, TCEP and the like. The buffer concentration may be between 0.1 mM to 200 mM.

In some embodiments, the at least one Cas protein is selected from Cas9, Cas12 and Cas13 and/or a nickase, a catalytic dead variant, or a catalytic dead variant conjugated to a enzymatic function, such as base editor, fluorescent protein, nuclease, transcriptional activator or repressor, epigenetic modifier, thereof.

In some embodiments, the genome editing composition further comprises double stranded or single stranded DNA. In certain embodiments, the length of the double stranded or single stranded DNA is between 50 bp to 10 kb. The concentration of the double stranded or single stranded DNA may for example be between 0.1 pmol/μL to 100 pmol/μL for homologous recombination.

In some embodiments, the genome editing composition further comprises a tracer configured to trace the injection efficiency and for enabling the calculation of the injected volume. A suitable tracer may for example be an aqueous solution of Lucifer yellow, particularly in a concentration of 0.1 mg/mL to 10 mg/ml. In certain embodiments, genome editing composition comprises a labelled tracrRNA being labelled with a suitable injection efficiency label, in particular a fluorescent label, such as Atto550. In particular, a ratio between unlabeled tracrRNA and labelled tracrRNA, which are comprised in the gRNA, may range from 1:10 to 10:1.

In some embodiments, the genome editing composition further comprises one or more of nanoparticles, such as fluorescent, magnetic, or functionalized nanoparticles, viruses, cell organelles, such as mitochondria, ribosomes, hormones, growth factors, inhibitors, proteins, peptides, amino acids, neurotransmitters, lipids, conjugates of lipids, proteins and/or sugars, RNA, small molecules and drugs.

In some embodiments, prior to direct injection of the genome editing composition, the cell is approached by the nanosyringe with an approach speed of 1 μm/s to 1 mm/s, which avoid cell damage. In particular embodiments, the approaching of the nanosyringe is under control of control unit ensuring a constant approaching speed.

In some embodiments, the force applied by the nanosyringe to penetrate the cell membrane is between 1 nN to 5000 nN, preferably between 700 nN to 1500 nN.

In order to avoid cell damage after injecting the genome editing composition, the nanosyringe is in some embodiments retracted with a speed of 1 μm/s to 1000 μm/s from the cell.

In some embodiments, the injection time for injecting the genome editing composition into the nucleus of the cell is between 1 s and 60 s.

In some embodiments, the genome editing composition is injected into the nucleus of the cell by applying a pressure of between 0 mbar and 5000 mbar with respect to atmospheric pressure to the microchannel of the cantilever.

In some embodiments, the microelectromechanical systems injection chip and/or the microelectromechanical systems transport chip is operationally connected to a positioning unit configured for positioning the microelectromechanical systems injection chip in the 3D space. The positioning unit may be an automatic positioning unit. It may also be under control of a control unit. The positioning unit may at least be realized by a set of axes, for example three orthogonal axes (x, y, z), thereby establishing a Cartesian kinematic robot structure. The x-axis and the y-axis are referred to as lateral axis and are, in an operational configuration levelled respectively traverse to the direction of gravity, while the z-axis is referred to as vertical axis and is aligned with the direction of gravity. The axes are typically motorized axes and realized, for example, as linear spindle or linear motor drives as generally known in the art. In some embodiments either of the lateral axes (e. g. the y-axis) is placed separately on a base support, while the other two axes (e.g., e. g. the x- and z-axis) are placed combined, with one axis carrying the other. In other embodiments, all three axes are placed as a combination (e.g. the y-axis carries the x-axis and the z-axis is carried by the x-axis).

In some embodiments, a laser beam is directed on the cantilever and a photodiode is arranged such that it collects the laser beam being reflected by the cantilever. A laser providing the laser beam and the photodiode can both be under control of the control unit.

In some embodiments, visual control may be achieved by a microscope, which may also be controlled by the control unit. The microscope may be equipped with fluorescence or confocal imaging means.

In some embodiments, wherein a volume of 1 to 1000 fL of the genome editing composition is directly injected into the nucleus of the cell.

In some embodiments, the at least one gRNA molecule comprises a tracrRNA being annealed with a crRNA. In such embodiments, the tracrRNA and the crRNA are typically not covalently linked. Such annealed gRNA is known to be less efficient in cleaving a target DNA as compared to sgRNA, which consists of a single RNA molecule comprising the sequence of the crRNA and tracrRNA. However, as the method according to the invention has a much higher genome editing efficiency and specificity (due to the fact that the traditional transfection barrier is dispensed with and that the components are directly delivered into the nucleus), the decreased efficiency due to the use of annealed gRNA is negligible. A strong advantage however is that because of the decreased efficiency of annealed gRNA as compared to sgRNA, the off-target rate is profoundly decreased, thereby decreasing the occurrence of off-target effects.

In some embodiments, the at least one gRNA molecule is a sgRNA which consists of, or which comprises, a single RNA molecule comprising the sequence of the crRNA and tracrRNA.

According to another aspect, the general objective is achieved by a genetically engineered cell obtained by the method according to any of the embodiments described herein.

1. An in vitro method for producing genetically engineered cells, the method comprising direct injection of a genome editing composition into a nucleus of a cell, the genome editing composition comprising at least one Cas protein and at least one gRNA molecule directed to a genomic location to be edited, wherein the injection is performed with a microelectromechanical systems injection chip comprising a cantilever, the cantilever comprising a microchannel being in fluid communication with a nanosyringe, and wherein the direct injection comprises providing a fluid communication between the microchannel and the nucleus of the cell by insertion of the nanosyringe into the nucleus of the cell and injecting the genome editing composition via the microchannel through the nanosyringe into the nucleus of the cell, thereby production a genetically-engineered cell.

2. The method according to claim 1, further comprising expanding the genetically engineered cell to generate a first monoclonal cell culture.

3. The method according to claim 2, wherein the cells of the first monoclonal cell culture are divided into a first sub-group and a second subgroup, wherein the cells of the first subgroup are analyzed by sequencing and wherein the cells of the second sub-group are expanded further to generate a second monoclonal cell culture.

4. The method according to claim 3, wherein the cells of the first monoclonal cell culture are additionally divided into a third sub-group, wherein the cells of the third sub-group are expanded further to produce a third monoclonal cell culture.

5. The method according to claim 1, wherein prior to direct injection of the genome editing composition, a cell preparation comprising a plurality of cells is provided and direct injection of the genome editing composition is performed on one or more cells of the cell preparation and wherein after direct injection of the genome editing composition, a single cell injected with the genome editing composition is selected and separated from the cell preparation.

6. The method according to claim 5, wherein selection and separation of the single cell comprises moving the single cell by a microelectromechanical systems transport chip comprising a cantilever with an opening and a microchannel extending through the cantilever to the opening, wherein the moving is performed by applying a negative pressure to the microchannel such that the single cell adheres to the cantilever.

7. The method according to claim 1, wherein prior to direct injection of the genome editing composition into the nucleus of the cell, a single cell is isolated.

8. The method according to claim 1, wherein the genome editing composition further comprises one or more transfection markers.

9. The method according to claim 1, wherein the genome editing composition comprises a plurality of Cas proteins and a plurality of gRNA molecules each directed to a genomic location to be edited.

10. The method according to claim 1, wherein the genome editing composition further comprises a buffer composition with one or more buffers with a pH range of 6 to 9, and/or inorganic salt mixtures.

11. The method according to claim 1, wherein the at least one Cas protein is selected from the group consisting of Cas9, Cas12, Cas13, a nickase, a catalytic dead variant, or a catalytic dead variant conjugated to an enzymatic function.

12. The method according to claim 1, wherein the genome editing composition further comprises double stranded or single stranded DNA for homologous recombination.

13. The method according to claim 1, wherein the genome editing composition further comprises one or more nanoparticles, viruses, cell organelles, hormones, growth factors, inhibitors, proteins, peptides, amino acids, neurotransmitters, lipids, conjugates of lipids, proteins, sugars, RNA, small molecules, and/or drugs.

14. The method according to claim 1, wherein prior to direct injection of the genome editing composition, the cell is approached by the nanosyringe with an approach speed of 1 μm/s to 1 mm/s.

15. The method according to claim 1, wherein the genome editing composition is injected into the nucleus of the cell by applying a pressure of between 0 mbar and 5000 mbar with respect to atmospheric pressure to the microchannel of the cantilever.

16. The method according to claim 1, wherein the microelectromechanical systems injection chip is operationally connected to a positioning unit configured for positioning the microelectromechanical systems injection chip in a 3D space.

17. The method according to claim 1, wherein a volume of 1 to 1000 fL of the genome editing composition is directly injected into the nucleus of the cell.

18. The method according to claim 1, wherein the at least one gRNA molecule comprises a tracrRNA annealed with a crRNA.

19. The method according to claim 1, wherein the at least one gRNA molecule is a sgRNA consisting of a single RNA molecule comprising the sequence of a crRNA and a tracrRNA.

20. A genetically engineered cell obtained by the method according to claim 1.
